(19) United States
(12) Patent Application Publication
(10) Pub. No.: US 2026/0026888 A1
(43) Pub. Date: Jan. 29, 2026

(54) TRACKING SURGICAL FRAME, NAVIGATION SYSTEM, AND NAVIGATION METHOD

(71) Applicant: B. Braun New Ventures GmbH, Freiburg im Breisgau (DE)

(72) Inventors: Amir Sarvestani, Freiburg (DE); Jean Stawiaski, Kirchzarten (DE)

(21) Appl. No.: 18/998,215

(22) PCT Filed: Jul. 19, 2023
(86) PCT No.: PCT/EP2023/070066

§ 371 Date: Jan. 24, 2025

(30) Foreign Application Priority Data
Jul. 26, 2022 (DE) 10 2022 118 714.4

(51) Int. Cl. A61B 34/20; A61B 34/30; A61B 90/00

(52) U.S. Cl. CPC A61B 34/20; A61B 34/30; A61B 90/39; A61B 2034/2057; A61B 2090/3979

(57) ABSTRACT

A tracking surgery frame for tracking a patient via a surgical navigation system includes an opening for entry to an intervention region, which is arrangeable around an intervention region and fixable with or to the patient so that a geometrical relationship between the intervention region and frame does not change. A first rigid body has at least one first marker for being tracked by the navigation system. A second rigid body has a second marker for being tracked by the navigation system. At least three markers are connected to the frame. The frame provides access to and a view of the intervention region via the opening. The first rigid body is spaced further apart from the second rigid body than the first rigid body is spaced apart from a portion of the frame. The frame can be used with a navigation system, navigation method and computer-readable storage medium.

### CROSS-REFERENCE TO RELATED APPLICATIONS

This application is the United States national stage entry of International Application No. PCT/EP2023/070066, filed on Jul. 19, 2023, and claims priority to German Application No. 10 2022 118 714.4, filed on Jul. 26, 2022. The contents of International Application No. PCT/EP2023/070066 and German Application No. 10 2022 118 714.4 are incorporated by reference herein in their entireties.

### FIELD

The present disclosure relates to a tracking surgery frame for a surgical intervention on a patient for tracking the patient via a surgical navigation system. In addition, the present disclosure relates to a navigation system, a navigation method as well as a computer-readable storage medium.

### BACKGROUND

Navigation is a technology used as standard in the field of neurosurgery. The head of a patient on which an intervention is being performed is usually fixed with a head support system or a head support. In order to localize/track the patient, a single patient tracker with a rigid body, which has several markers/reference points defined in relation to each other in space, is usually attached to this head support. The patient tracker is usually attached to a single (lateral) side of the head of the patient, to the left or right of it. This approach described above is also used in robotic neurosurgery. In these areas, accuracy is particularly important for a successful intervention.

However, in terms of the required accuracy, in most cases the patient tracker is the weakest link in a system that is as precise as possible. One reason for this is the geometric positioning of the patient tracker relative to the intervention region or to the anatomy to be operated on, for example the brain. Due to a forced distance between the patient tracker on the one hand and the anatomy of interest on the other hand, on which the intervention is carried out and on which the operation is performed, there is a large lever arm, so to speak, which causes a proportional amplification of small deviations on the patient tracker when a point is localized in or on the anatomy to be operated on by the navigation or robotic system.

Even under ideal conditions, this circumstance may lead to inaccuracies of 1 mm or more on the anatomy to be operated on, which in particular in combination with other deviations is usually no longer tolerable. A further contribution to inaccuracy is caused by the fact that the patient tracker has to be exchanged in the portion after a registration and before an operation, since a device/frame is not sterile during registration, but the device must definitely be sterile during the operation. This lack of sterility during registration is unfortunately necessary, since optical access to a patient's face is required, which serves as a reference for the registration. During the operation/intervention, however, the face is covered.

Replacing the patient tracker may in turn lead to inaccuracies of 2 mm or more in relation to the anatomy of the patient. In order to counter this limitation, very large individual patient trackers have been developed according to the state of the art, which, however, each have an extension of 15 cm or more. However, this large geometry adversely affects the surgical field and may itself be a further source of inaccuracies, for example if the tracker with large configuration is inadvertently touched by medical or surgical staff.

A similar problem of inaccuracy of a patient tracker arises in the area of spine surgery, in which the patient tracker is attached to a predetermined spine-bone structure with a cantilever arm to the anatomy to be operated on.

### SUMMARY

It is therefore the object of the present disclosure to avoid or at least reduce the disadvantages of the prior art and in particular to provide a tracking surgery frame as a patient tracker, a navigation system and a navigation method which allows precise and repeatable tracking and registration of a patient in a particular way. In particular, a partial object is to ensure particularly robust tracking with tracking components with small configuration. In particular, an error in registration and tracking is to be minimized, even when markers used for tracking are replaced.

The objects are solved with respect to a generic tracking surgery frame according to the invention, with respect to a generic navigation system according to the invention, with respect to a generic navigation method according to the invention and with respect to a computer-readable storage medium according to the invention.

Thus, a basic idea of the present invention provides that a (patient) tracking surgery frame is provided as a patient tracker for a surgical navigation via a navigation system, wherein the tracking surgery frame surrounds an anatomy of interest by providing two or more independent rigid bodies/reference markers/trackers. These may be combined to calculate and/or create a combined patient tracker/combination patient tracker in order to increase the accuracy of navigation and robotics.

Therefore, if a rigid body/tracker moves slightly due to mechanical tolerances, or due to an exchange of trackers or unintentional touching by surgical staff, rotational movements of the rigid body (around a connection point) are usually caused, which have a significant negative influence on the accuracy due to the leverage effect, while translational movements of the rigid body are often negligible. However, the combination of two rigid bodies and the corresponding configuration of the tracking surgery frame avoids the leverage effects during rotational movements and consequently does not lead to increased inaccuracies in the anatomy to be operated on.

In other words, an extended patient tracker/tracking surgery frame is proposed in which the markers/reference points for registration are arranged (spaced) around the intervention region, in particular around a head of the patient, by the navigation system. In this way, small deviations between the markers are not amplified and do not lead to inaccuracies in the surgical field. A “leverage effect” of a first rigid body is compensated in particular by an opposing “leverage effect” of the second rigid body, which is arranged as far away as possible from the first rigid body, and an error can thus be minimized.

In particular, the tracking surgery frame (as an extended patient tracker) may be used for navigated and robot-assisted surgery using an optical navigation system. For this purpose so to speak, a first patient tracker is attached to the (surgical) frame, in particular to a head support, and has or consists of a regular rigid body with at least one marker/reference point/fiducial mark, in particular with three markers. A further, separate, second patient tracker with one or more markers/reference points (active or passive) is additionally attached to the (surgical) frame. The two trackers or rigid bodies with markers are possibly arranged in such a way that the entirety of the markers/fiducial markers geometrically surrounds the intervention region or the anatomy to be operated on, e.g. the head of the patient. It can also be said that the tracking surgery frame has such a design that at least three markers are arranged around an opening (towards an intervention region), in particular equally distributed. In particular, the rigid body with markers (as individual patient trackers) may be attached to the head support, as used in neurosurgery, such that the combined markers surround the intervention region/surgical field and are visible to a navigation camera of a navigation system.

With the help of a control unit, a new combined rigid body (combination patient tracker) can be created from the combination of all markers/fiducial marks of the at least two trackers (first and second rigid body) that can be tracked or are tracked by the navigation camera of a navigation system. In order to do this, the trackers/rigid bodies are first tracked separately and then combined into a new, integrated tracker.

In yet other words, the present disclosure proposes a tracking surgery frame (as an extended patient tracker) for a surgical intervention on a patient for tracking the patient via a surgical navigation system. The tracking surgery frame has a central (entry) opening or recess for entry to an intervention region. The tracking surgery frame may also be said to be configured to extend at least partially around an intervention region as a base frame, but still leaving an entry to the intervention region or surgical field to be used intraoperatively.

The tracking surgery frame is therefore arrangeable around an intervention region, for example a skull opening during a craniotomy, and is rigidly fixable with or to the patient P. With this fixation, the geometric relationship between the intervention region and the tracking surgery frame does not change during the intervention, and a relationship always remains static or rigid. The tracking surgery frame has a first rigid body/tracker with at least one first marker/reference element for being tracked by a navigation system, in particular a navigation camera of a navigation system. The rigid body is rigidly connected to the tracking surgery frame, in particular non-detachably attached or detachably coupleable and uncoupleable.

Furthermore, the tracking surgery frame has a second rigid body/tracker (separate and distinct from the first), which has at least one second marker for being tracked by a navigation system and which is rigidly connected to the tracking surgery frame. In total, however, at least three markers are connected to the tracking surgery frame via the at least two rigid bodies in order to allow clear referencing and tracking in space.

The tracking surgery frame with the connected rigid bodies is configured in such a way that it provides access to and a view of the intervention region via the central opening or recess, i.e. can be used intraoperatively, and the first rigid body, in particular the at least one first marker, is spaced further apart/further away from the second rigid body, in particular from the at least one second marker, than the first rigid body, in particular the at least one first marker, is spaced apart from a portion of the tracking surgery frame which is closest to the first rigid body, in particular to the first marker, and which forms the opening or recess to the intervention region. In particular, the markers are to be arranged as far apart as possible around the intervention region in order to ensure the most accurate tracking possible and minimization of errors.

In contrast to the prior art, not only a rigid body/tracker with markers is provided locally in only a very small spatial volume on the patient, which, when the rigid body moves, increases an error proportionally due to a lever arm and would lead to an unacceptable inaccuracy, but markers are provided distributed around the intervention region, which can be combined together during navigation via a navigation system to create a combination patient tracker.

In particular, the relationship of the first rigid body to the second rigid body and thus of the first marker to the second markers is known.

Advantageous embodiments are explained in particular below.

According to one embodiment, the tracking surgery frame may be configured in the form of a head support for a neurosurgical intervention on the brain. This head support then preferably has the various rigid bodies with markers around the head of the patient. Alternatively, the tracking surgery frame may be configured in the form of a spinal frame, which is detachably attachable to spinous processes of the spine, which is trackable/can be tracked (spatially) via the spaced rigid bodies with markers. Due to the configuration of the present disclosure, a surgeon has easy access to the intervention region and the navigation camera also has a good view of the markers of the tracking surgery frame. There are no large components in terms of installation space, which would make an intervention more difficult, but only two trackers spaced apart from each other are used. In spine surgery, the tracking surgery frame with the two trackers may be attached to bony structures of the spine in such a way that the combined markers/reference points surround the surgical field and are visible to a navigation camera.

According to a further embodiment, based on this in the case of the head support, the first rigid body may be arranged on a lateral side of the patient, in particular on the dexter side or on a right side, and the second rigid body may be arranged on the opposite lateral side, in particular on the sinister side or on a left side, in order to achieve a particularly large distance between the first marker and the second marker. A center of gravity of the combined common markers thus lies in the intervention region.

In particular, in a top view of the central opening or recess surrounding the intervention region (i.e. in a view perpendicular to the intervention region/the anatomy to be operated on) around the central opening or recess on a virtual circle or circularly, in particular with a center of the circle near or in a center of the opening or recess, which in particular also corresponds to a center of the intervention region, the at least first and second rigid bodies with the markers may be arranged on the virtual circle, in particular in an equally distributed manner. This ensures the best possible “framing” of the intervention region with markers, which further increase precision due to their arrangement.

Preferably, in the case of a virtual circle, in the case of exactly two rigid bodies with markers, these may lie substantially diagonally opposite each other with respect to the opening or recess and may form a virtual straight line through the intervention region in connection with each other, in the case of exactly three rigid bodies with markers, these may form a virtual, in particular equilateral, triangle in connection with each other with respect to the opening or recess, in the case of exactly four rigid bodies with markers, these may form a virtual rectangle, in particular square, in connection with each other with respect to the opening or recess. These special arrangements, depending on the number of rigid bodies, offer particularly good precision. The number of markers may of course differ from rigid body to rigid body, as long as more than three markers are arranged in total, preferably more than three markers per rigid body.

According to a further embodiment, passive infrared markers and/or active infrared LEDs and/or optical patterns, such as QR codes in particular, may be used as markers. In particular, the markers/reference points may be passive markers (e.g. NDI) and/or active LEDs.

Preferably, the first rigid body and/or second rigid body may have a (mechanical) adapter/coupling structure adapted to decouple the rigid body from the tracking surgery frame and to couple it again in a releasable and accurately repeatable manner, so that the rigid body with the at least one marker/reference point may be exchanged during the operation without changing a position of the at least one marker/reference point. In other words, the rigid bodies of the tracking surgery frame may preferably have an adapter so that they can be exchanged with repeat accuracy during the operation.

Preferably, the adapter is adapted to be coupled and uncoupled without tools to ensure a quick but accurately repeatable intraoperative change. Preferably, the adapter has a button that a user can actuate manually and a locking mechanism that is in a locked state without actuation of the (manual) button, in which the adapter is not releasably coupled, and changes to an unlocked state against pre-stressing when the button is actuated in order to decouple the adapter from the tracking surgery frame. This provides the user with a simple but efficient mechanism with a quick-release button that allows the rigid body to be coupled and uncoupled safely and without tools.

In particular, the first and second rigid bodies are each spaced apart from the surgery frame, and preferably have a distance of more than 5 cm, particularly preferably more than 10 cm, and very particularly preferably more than 20 cm.

In particular, the tracking surgery frame may be configured in the form of a head support for a neurosurgical intervention on the brain with skull pins, which has a U-shaped base structure wherein a single cranial pin (skull pin) is provided on one cantilever arm of the U-shaped base structure, wherein the skull pin points toward the opposite cantilever arm on which in turn two skull pins are arranged (i.e. a total of three skull pins). The two skull pins point toward the one skull pin and are also (as they are not parallel, slightly) facing each other. In this way, a skull can be defined in space via the three spaced skull pins. In particular, the head support may be formed as a radiolucent skull clamp (e.g. metal-free, for example with plastic as the material). In particular, the first and second rigid bodies are arranged diametrically opposite the head support, in particular the U-shaped base structure (opposite each other) (one could also say laterally facing away from each other at a greater distance from each other than a dimension of the head support) and are each at a distance from the head support.

In particular, the navigation system has a robot which has a visualization unit or a manipulator as an end effector.

Preferably, the first rigid body and/or the second rigid body have one, two, three, four or more markers/reference points, wherein the rigid bodies of the tracking surgery frame have a total of at least three reference points. Each tracker may have one, two, three, four or more markers/fiducial marks, wherein the total number of markers/fiducial marks has to be at least three.

The objects of the present disclosure are solved with respect to a navigation system with registration and navigation function for a surgical intervention on a patient by comprising: a visual display device for visual navigation, in particular a surgical monitor, a VR headset or a display; a navigation camera, in particular a 3D navigation camera, particularly preferably a stereo camera, for optical detection and tracking of markers; a control unit specially adapted to process the optical detection of the navigation camera, to perform registration of the patient and navigation functions, and to output navigation guidance via the visual display device. The navigation system according to the present disclosure further comprises a rigid tracking surgery frame according to the present disclosure, wherein the tracking surgery frame comprises a total of at least three markers (of the first rigid body and of the second rigid body). In addition, the control unit is further adapted to determine a combination patient tracker based on the first rigid body of the tracking surgery frame with the at least first marker and the second rigid body with the at least second marker, i.e. to combine the markers, and the navigation system performs a registration based on the combination patient tracker. A navigation camera may be used to localize each marker in space and to correlate them with each other via the control unit.

According to an embodiment of the navigation system, the control unit may determine a single combination patient tracker with all of the at least three markers based on the at least three markers of the tracking surgery frame. The control unit thus uses all markers of the tracking surgery frame and creates a single (virtual) combination patient tracker, which may be used for registration and in particular for being tracked by the navigation system. All in particular all markers/reference points of both rigid bodies are used to create a new virtual combination patient tracker

According to a further embodiment, the control unit may calculate a first transformation matrix for registration of the patient based on the first rigid body with the at least one first marker, and may calculate a second transformation matrix for registration of the patient based on the second rigid body with the at least one second marker, and may calculate an averaged transformation matrix for the combination patient tracker based on the first and second transformation matrix in order to minimize an error in registration and tracking via redundancy. In particular, the two trackers are tracked separately and then a new reference is created by averaging.

Preferably, the control unit may be adapted to detect a change in position of a marker (relative to other markers) of the tracking surgery frame relative to an (in particular initially) determined combination patient tracker, and to output a warning message via the visual display device. In this way, the combination patient tracker may provide an important safety feature, i.e. the detection of a displacement of a single marker relative to the other markers. The arrangement of the markers always is to be static and maintain an unchanging relationship between the markers. The (virtually) created combination patient tracker serves as a basis for comparison. If, for example, a surgical staff member touches a marker and moves it unintentionally, this can be detected by the navigation system and a corresponding warning, in particular also an automatic adjustment of tracking, may be carried out. The navigation system not only increases the accuracy of surgical navigation, but also makes it possible to recognize whether a tracker (with its marker) has moved after registration, since the relative position between the trackers is continuously tracked by the navigation system. In this way, the user may be informed of any interference in accuracy due to a change in position of a marker.

Preferably, the control unit may be adapted to perform weighting for the first rigid body and the second rigid body in order to shift a center of gravity of the combination patient tracker toward the intervention region, in particular in antiproportional dependence on the number of markers on the rigid body. For example, if there is only a single marker on the first rigid body and four markers on the second rigid body, a center of gravity would be on the side of the second rigid body without weighting. A change or movement of the second rigid body would have a much greater effect than a change in the first rigid body. However, it can be assumed that the first rigid body should compensate for a possible error of the second rigid body, which is why the first rigid body is to be assigned a correspondingly higher weighting. This may also be seen graphically in such a way that a common center of gravity of the marker is to be set as central as possible to the opening of the tracking surgery frame or to the intervention region, so that the best possible error correction or the best possible, robust, precise combination patient tracker is achieved. It can also be said that the control unit uses a weighting for the markers/reference points in order to minimize an error in a middle field of the reference points in which the intervention is to be performed. In particular, special weighting algorithms may be used to weight the markers/reference points in such a way that the accuracy of a midpoint of the markers/reference points is maximized.

In particular, the control unit may be adapted to perform weighting of the individual rigid body that is antiproportional to the markers in accordance with the formula weighting (rigid body)=1/number of markers (rigid body). For example, if the first rigid body has one marker, the second rigid body has 4 markers and a third rigid body has two markers, the weighting is as follows

$$weighting ( rigid body 1 ) = 1 / 1 = 1$$

$$weighting ( rigid body 2 ) = 1 / 4 = 0 \text{.25}$$

$$weighting ( rigid body 3 ) = 1 / 2 = 0.5$$

This shifts the center of the weighted rigid body or markers into the intervention region.

In particular, the navigation system may have other trackers in addition to the tracking-surgery frame, such as instrument trackers, which preferably each have three or four markers.

Preferably, the control unit may be adapted to perform a measurement of the relative pose between the at least first and second rigid body with the navigation camera and may be adapted to detect an inaccuracy or deviation for the navigation.

The objects are solved with respect to a navigation method for registration of a patient and navigation via a tracking surgery frame with at least a first and a second rigid body with respective corresponding markers, according to the present disclosure by the steps of: detecting markers of the first rigid body; detecting markers of the second rigid body (wherein it is irrelevant whether the first or second rigid body is detected first); calculating and creating a combination patient tracker by a control unit; performing a registration based on the combination patient tracker; and preferably performing a navigation based on and in relation to the combination patient tracker. The individual rigid bodies, for example two or three standardized rigid bodies, are first detected and a combination patient tracker is calculated or created based on the individual rigid bodies. This is then used for registration.

Preferably, the navigation method may further comprise the step of:
detecting a movement or displacement of individual markers of the combination patient tracker, and preferably issuing a warning message via a display device to a user when a change in the markers arranged rigidly relative to each other has been detected. The navigation method recognizes unwanted displacements and thus sources of errors and warns the user accordingly.

With regard to a computer-readable storage medium, the object is solved in that it comprises instructions which, when executed by a computer, cause the computer to execute the method steps according to the navigation method of the present disclosure.

In particular, the navigation method may comprise the following steps: arranging a first patient tracker; arranging a second patient tracker; tracking the first patient tracker; tracking the second patient tracker; calculating and creating a combined rigid body as a tracker; performing a registration based on the combined tracker; performing a navigation based on/in relation to the combined tracker; detecting a movement or displacement of individual trackers;

The disclosure regarding the tracking surgery frame according to the present disclosure applies also to the navigation system and the navigation method of the present disclosure and vice versa.

### BRIEF DESCRIPTION OF THE DRAWINGS

The present disclosure is explained in more detail below with reference to the accompanying Figures, with reference to preferred embodiments. The following is shown:

FIG. 1 shows a perspective view of a tracking surgery frame in the field of neurosurgery according to a first preferred embodiment, which is used in a navigation system according to a preferred embodiment;

FIG. 2 shows a perspective view of a tracking surgery frame according to a further preferred embodiment, which is used in the area of spine surgery in a navigation system according to a further preferred embodiment;

FIG. 3a shows a schematic view explaining how a combination patient tracker is created from two individual rigid bodies;

FIG. 3b shows a further schematic view explaining another way of creating a combination patient tracker from two individual rigid bodies;

FIG. 4 shows a schematic view of a head support as a tracking surgery frame with two rigid bodies;

FIG. 5 shows a perspective view of a navigation system of a further preferred embodiment, which uses further trackers; and

FIG. 6 shows a flowchart of a navigation method according to a preferred embodiment.

The Figures are schematic in nature and are only intended to aid understanding of the invention. Identical elements are marked with the same reference signs. The features of the various embodiments may be interchanged.

### DETAILED DESCRIPTION

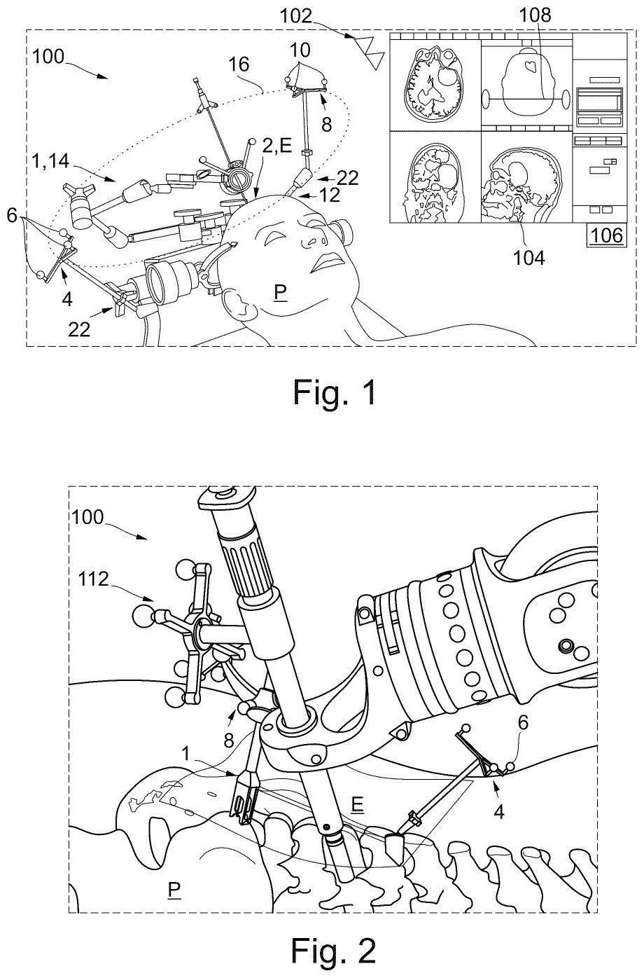

FIG. 1 shows a perspective view of a tracking surgery frame 1 according to a first preferred embodiment for a surgical intervention on a patient P for tracking the patient P via a surgical navigation system 100 in the area of a craniotomy according to a preferred embodiment.

In the present case, the tracking surgery frame 1 (hereinafter referred to only as surgery frame) has a central opening 2 for entry to an opening in the skull of the patient or to the brain as intervention region E. The surgery frame 1 is arranged with its opening 2 around this intervention region E in sections and is rigidly fixed to both an operating table and to the patient P. In this way, the geometric relationship between the intervention region E and the tracking surgery frame 1 cannot change and the relationship remains static. It can also be said that the head of the patient may be inserted into the opening 2 of the surgery frame 1 of the opposite C-shaped fixation claws and may be fixed later.

The surgery frame 1 has a first rigid body 4 (a first patient tracker arranged on a cantilever) with three first markers 6 for being tracked by a navigation camera 102 of the navigation system 100 and is rigidly attached to the surgery frame 1.

Furthermore, the surgery frame 1 has at least one second rigid body 8 with at least three second markers 10 for being tracked by the navigation system 100, which is arranged opposite the first rigid body on the other side of the head of the patient. The second rigid body 8 is also rigidly attached to the surgery frame 1. This means that a total of six markers are attached to the surgery frame 1, i.e. more than three markers (6, 10) are required for clear tracking.

The surgery frame 1 with the connected rigid bodies 4, 8 is designed in such a way that it provides access and a view of the intervention region E at the posterior cranial region via the central opening 2. According to the present disclosure, the first rigid body 4 with its three first markers 6 is spaced further apart from the second rigid body 8 with its three second markers 10 than the first rigid body 4 is from the closest portion 12 of the surgery frame 1 that forms the opening 2 to the intervention region E. Due to this design of two rigid bodies 4 and 8 located opposite each other relative to the intervention site E or the opening 2, markers may be arranged around the intervention region E opposite each other and widely spaced.

In this embodiment, the surgery frame 1 is configured in the form of a head support 14 for a neurosurgical intervention on the brain. FIG. 1 also shows a virtual circle 16 as a reference circle. The configuration is in particular such that in a plan view of the intervention region E, around the central opening 2 and the intervention region E on the virtual circle 16, the center 18 of the circle of which lies in a center of the opening 2, the first and second rigid bodies 4, 8 with the markers 6, 10 are arranged on the virtual circle (16) with equal distribution, i.e. diagonally opposite each other.

Finally, in order to be able to use the surgery frame 1 for navigation, the navigation system 100 has a registration and navigation function for a surgical intervention on a patient P. As hardware components, the navigation system has a visual display device 104 in the form of a surgical monitor to provide visual navigation.

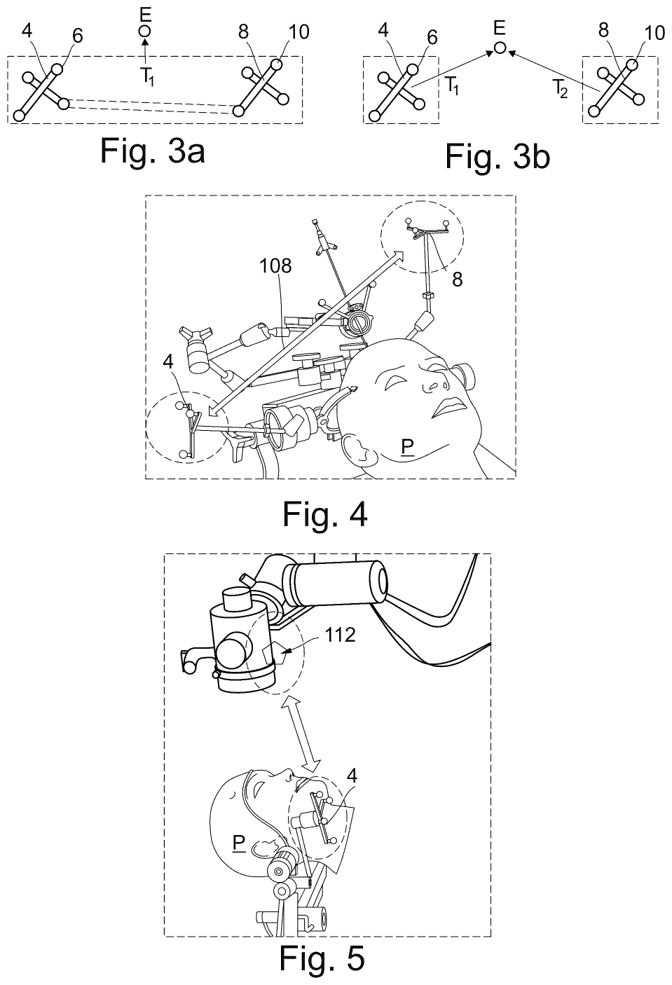

Furthermore, the navigation system 100 comprises a navigation camera 102 for optically detecting and tracking the markers 6, 10, and a control unit 106, such as a computer system, which is adapted to process the optical detecting of the navigation camera 102, to perform a registration of the patient P and navigation functions and to output navigation guidance via the visual display device 104. Specifically, the control unit 106 is adapted to determine a combination patient tracker 108 based on the first rigid body 4 with the three first markers 6 and the second rigid body 8 with the three second markers 10, and to perform a registration based on the combination patient tracker 108. Specifically, the control unit 106 forms a single combination patient tracker 108 based on the six markers 6, 10 of the surgery frame 1 with all of the at least three markers 6, 8 of the patient surgery frame 1. The detailed creation and mode of operation will be explained in more detail later with respect to FIG. 3a.

The combination patient tracker 108 can minimize errors due to possible displacements and can further increase the precision of navigation.

FIG. 2 shows a perspective view of a further embodiment of a surgery frame 1, which is used in the field of spine surgery.

The functionality and configuration of the navigation system 100 is similar to that shown in FIG. 1, with the difference that the surgery frame 1 is configured differently and is adapted for connection to a spinous process of the spine. The markers may again be arranged outside and around the actual intervention region via the first and second rigid bodies 4 and 8. The first rigid body 4 and the second rigid body are connected to each other.

FIGS. 3a and 3b show two alternative possibilities of a creation of a combination patient tracker 108 with respect to a target area, which represents an intervention region E.

In FIG. 3a, all markers 6, 10 of the surgery frame 1 are used and a virtual large combination patient tracker 108 with all markers 6, 10 is created. The rigid bodies 4 and 8 themselves no longer play a role and are replaced by the combination patient tracker 108. This combination patient tracker 108 is then used by the navigation system 100 for registration.

FIG. 3b shows another alternative of creation of a combination patient tracker 108. In this case, the individual rigid bodies 4 and 8 are used, and an average value of a synopsis of the two rigid bodies 4 and 8 is created, so to speak. This quasi averaged rigid body can be referred to as combination patient tracker 108 and may be used for registration.

FIG. 4 shows a perspective schematic view of the surgery frame 1 from FIG. 1 in a more detailed view. According to the above view and creation of the combination patient tracker 108, this is also virtually seen by the navigation system as indicated in FIG. 4, and the navigation system 100 may carry out corresponding registration and tracking.

FIG. 5 shows a further perspective view of a navigation system 100 according to a further embodiment, in which further trackers are integrated, such as robot trackers 110 or instrument trackers (not shown).

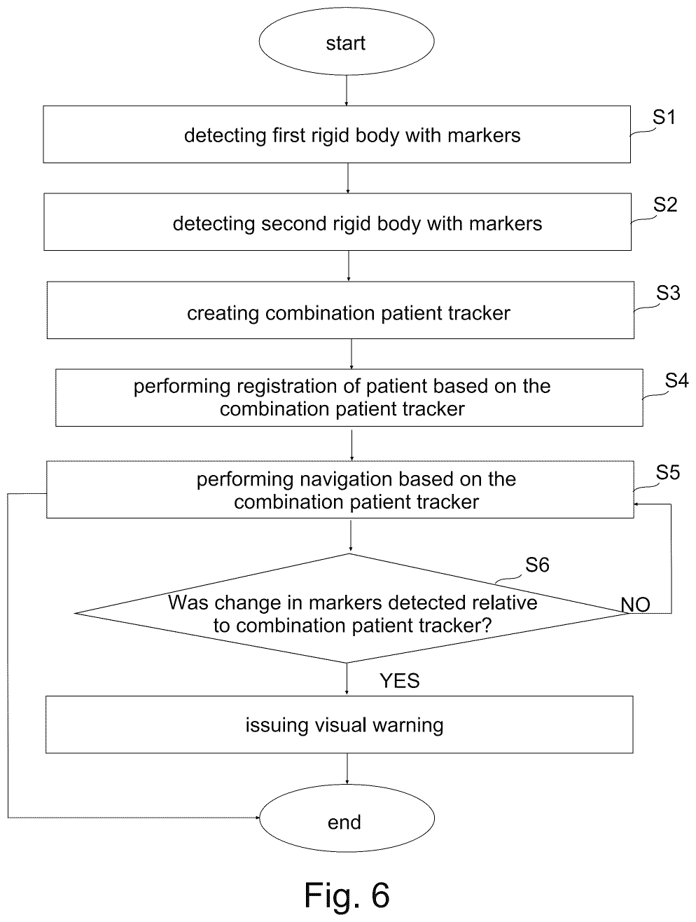

FIG. 6 shows a flowchart of a navigation method according to the disclosure for registration of a patient P and navigation via a tracking surgery frame 1 with at least a first rigid body 4 and a second rigid body 8 with respective corresponding markers 6, 10 by a navigation system 100. The navigation method comprises the following steps.

In a first step S1, markers 6 of the first rigid body 4 of the surgery frame 1 are detected S1 by a navigation camera 102 of a navigation system 100.

In a subsequent step S2, the markers 10 of the second rigid body of the surgery frame 1 are then detected.

In step S3, a combination patient tracker 108 is calculated and created by a control unit 102, which combines all markers 6, 8 of the surgery frame 1 into a single combination patient tracker 108.

After this, a registration based on the combination patient tracker 108 is carried out in step S4.

After registration, navigation based on and in relation to the combination patient tracker 108 takes place in step S5.

During navigation, step S6 is performed: detecting a movement or displacement of the individual markers 6, 10 of the surgery frame 1 with respect to the combination patient tracker 108, and in step S7 issuing a warning message via a display device 104 to a user when a change in the rigidly arranged markers 6, 10 has been detected.

### LIST OF REFERENCE SIGNS

1 tracking surgery frame
2 central opening
4 first rigid body
6 first marker
8 second rigid body
10 second marker
12 portion
14 head support
16 virtual circle
18 center of the circle
20 virtual straight line
22 adapter
100 surgical navigation system
102 navigation camera
104 display device
106 control unit
108 combination patient tracker
110 robot tracker
P patient
E intervention region

1.-16. (canceled)

17. A navigation system with registration and navigation function for a surgical intervention on a patient, the navigation system comprising:
a visual display device for visual navigation;
a navigation camera for optical detection and tracking of markers;
a control unit adapted to process the optical detection of the navigation camera, to perform a registration of patient and navigation functions, and to output navigation guidance via the visual display device; and
a tracking surgery frame,
the tracking surgery frame being rigid and comprising:
a central opening or recess for entry to an intervention region, the central opening being arrangeable around an intervention region and being rigidly fixable with or to the patient, so that a geometrical relationship between the intervention region and the tracking surgery frame does not change,
a first rigid body with at least one first marker for being tracked by the navigation camera, the first rigid body being rigidly connected to the tracking surgery frame; and
at least one second rigid body with at least one second marker for being tracked by the navigation camera, the at least one second rigid body being rigidly connected to the tracking surgery frame,
a total of at least three markers of the first rigid body and of the at least one second rigid body being connected to the tracking surgery frame,
the tracking surgery frame, the first rigid body and the at least one second rigid body being configured to provide access to and a view of the intervention region via the central opening or recess,
a distance between the first rigid body and the at least one second rigid body being greater than a distance between the first rigid body and a portion of the tracking surgery frame which is closest to the first rigid body and which forms the central opening or recess to the intervention region,
the control unit being further adapted to determine a combination patient tracker based on the first rigid body with the at least one first marker and the at least one second rigid body with the at least one second marker, the tracking surgery frame comprising at least three markers in total, and the navigation system being adapted to perform a registration based on the combination patient tracker.

18. The navigation system according to claim 17, wherein the tracking surgery frame is configured as a head support for a neurosurgical intervention on the patient's brain that is trackable via the first rigid body and the at least one second rigid body with markers.

19. The navigation system according to claim 18, wherein the first rigid body is arranged on a lateral side of the patient, and the at least one second rigid body is arranged on an opposite lateral side, in order to achieve a large distance between the at least one first marker and the at least one second marker.

20. The navigation system according to claim 17, wherein the tracking surgery frame is configured as a spinal frame that is detachably attachable to spinous processes of the patient's spine, and that is trackable via the first rigid body and the at least one second rigid body with markers.

21. The navigation system according to claim 17, wherein, in a top view of the central opening or recess surrounding the intervention region, around the central opening or recess on a virtual circle, the first rigid body and the at least one second rigid body with the markers are arranged in an equally distributed manner on the virtual circle.

22. The navigation system according to claim 21, wherein the first rigid body and the at least one second rigid body total two rigid bodies that lie diagonally opposite each other with respect to the central opening or recess and form a virtual straight line through the intervention region in connection.

23. The navigation system according to claim 21, wherein the first rigid body and the at least one second rigid body total three rigid bodies that form a virtual triangle in connection with each other with respect to the central opening or recess.

24. The navigation system according to claim 21, wherein the first rigid body and the at least one second rigid body total four rigid bodies that form a virtual rectangle in connection with each other with respect to the central opening or recess.

25. The navigation system according to claim 17, wherein passive infrared markers and/or active infrared LEDs and/or optical patterns are used as markers.

26. The navigation system according to claim 17, wherein:
the first rigid body has a first adapter so that the first rigid body is decoupleable and recoupleable with the at least one first marker during operation and is exchangeable without changing a position of the at least one first marker; and/or
the at least one second rigid body has a second adapter so that the at least one second rigid body is decoupleable and recoupleable with the at least one second marker during operation and is exchangeable without changing a position of the at least one second marker.

27. The navigation system according to claim 17, wherein the combination patient tracker comprises a single combination patient tracker that the control unit determines based on all of the at least three markers.

28. The navigation system according to claim 17, wherein the control unit is configured to:
calculate a first transformation matrix for registration of the patient based on the first rigid body, wherein the at least one first marker comprises at least three first markers,
calculate a second transformation matrix for registration of the patient based on the at least one second rigid body, wherein the at least one second marker comprises at least three second markers, and
calculate an averaged transformation matrix for the combination patient tracker based on the first transformation matrix and the second transformation matrix for registration of the patient in order to minimize a registration error via redundancy.

29. The navigation system according to claim 17, wherein the control unit is adapted to detect a change in position of a marker of the combination patient tracker, and to output a warning message to a user via the visual display device.

30. The navigation system according to claim 17, wherein the control unit is adapted to perform weighting for the first rigid body and the at least one second rigid body in order to shift a center of gravity of the combination patient tracker toward the intervention region.

31. The navigation system according to claim 30, wherein the control unit is adapted to perform weighting of one of the first rigid body and the at least one second rigid body that is antiproportional to the markers.

32. A navigation method for registration of a patient and navigation via a tracking surgery frame with a first rigid body having at least one first marker and at least one second rigid body with at least one second marker, the navigation method comprising the steps of:
detecting the at least one first marker of the first rigid body;
detecting the at least one second marker of the at least one second rigid body;
calculating and creating a combination patient tracker by a control unit, and
performing a registration based on the combination patient tracker.

33. The navigation method according to claim 32, further comprising the step of detecting a movement or displacement of individual markers of the tracking surgery frame relative to the combination patient tracker.

34. The navigation method according to claim 33, further comprising the step of issuing a warning message via a display device to a user when a change in the markers arranged rigidly relative to each other has been detected.

35. The navigation method according to claim 32, further comprising the step of performing a navigation based on and in relation to the combination patient tracker.
